(19) United States
(12) Patent Application Publication
(10) Pub. No.: US 2018/0318279 A1
(43) Pub. Date: Nov. 8, 2018

(54) TOPICAL COMPOSITIONS PROVIDING PAIN RELIEF AND METHODS OF USE THEREOF

(71) Applicant: RUSH UNIVERSITY MEDICAL CENTER, CHICAGO, IL (US)

(72) Inventor: Kathleen Heneghan, Chicago, IL (US)

(73) Assignee: RUSH UNIVERSITY MEDICAL CENTER, CHICAGO, IL (US)

(21) Appl. No.: 15/769,901

(22) PCT Filed: Oct. 14, 2016
(86) PCT No.: PCT/US16/57078

§ 371 Date: Apr. 20, 2018

Related U.S. Application Data
(60) Provisional application No. 62/245,511, filed on Oct. 23, 2015.

(51) Int. Cl. A61K 31/451; A61K 9/00; A61K 47/10; A61K 47/14; A61K 47/38; A61P 29/00

(52) U.S. Cl. CPC A61K 31/451; A61K 9/0014; A61P 29/00; A61K 47/14; A61K 47/38; A61K 47/10

(57) ABSTRACT

The present invention generally relates to a topical composition including loperamide and a pharmaceutically acceptable carrier and to a method of using such a composition. One embodiment of the method provides for analgesia or antihyperalgesia.

### CROSS-REFERENCE TO RELATED APPLICATIONS

The present patent application claims the benefit of the filing date of U.S. Provisional Patent Application No. 62/245,511, filed Oct. 23, 2015, the contents of which is hereby incorporated by reference.

### TECHNICAL FIELD

The present invention generally relates to a topical composition including loperamide and a pharmaceutically acceptable carrier and to a method of using such a composition. One embodiment of the method provides for analgesia or antihyperalgesia.

### BACKGROUND

Lancing the finger or heel for blood specimen collection is a common procedure.¹⁻² The lancet, composed of a blade and spring load devise directly splices into the dermal layer to reach the capillaries while preventing penetration to the bone.² The fingertips in adults and the heel in infants are the most common lancing sites due to rich perfusion, yet there is also dense innervation, with the highest concentration of free nerve endings occurring in the fingertips.²⁻³ Thus lancing results in a sharp, pain response, along with hyperalgesia from the release of chemical inflammatory mediators.²⁻¹³ The pain from repeat fingertip lancing is a major reason for non-compliance with glucose testing in the diabetic population and a significant cause of physiologic distress and long term exaggerated pain response in the newborn population. Topical analgesics such as amethocaine gel, lignocaine ointment or topical lidocaine-prilocaine (EMLA), are not typically used in the adult and ineffective to alleviate the pain and physiologic distress during infant heel lancing.¹⁴⁻²¹ Considering nearly 10% of the US population is diabetic (28.9 million adults) 1 and 15% of the newborn population is high risk and requires on average 39 lancings per hospitalization (range 1-171) in a very limited are of the heel,⁴ there is a strong need to find a safe localized drug that decreases the pain experienced with lancing.

With the recent discovery of opioid receptors and opioid peptides on sensory peripheral neurons, opioid agonists (morphine, fentanyl, loperamide) have demonstrated a significant analgesia when locally injected or topically applied to a site of inflammation/injury. Peripheral opioids reduce nociceptor excitability the propagation of action potentials through their effects on calcium potassium and sodium channels as well as through glutamate receptors and G-protein coupled receptors (GPCR) (Table 1).²²⁻³³ Opioid receptor numbers are markedly up-regulated in the periphery only in inflammatory states, where tissue damage results in increased permeability of the perineural barrier and receptor availability within 10-30 minutes of tissue damage.³⁰⁻³¹ The opioid receptors extend along the sensory neurons and are found on C- and Aδ-fibers, vanniloid receptor-1-positive visceral fibers and on neurons expressing isolectin B4, substance P and calcitonin-gene-related peptide.²³⁻³⁴ This increased expression of opioid receptors as a result of inflammation suggests that peripherally administered opioids may be effective analgesics for certain types of injuries.

### SUMMARY OF THE PREFERRED EMBODIMENTS

In one aspect, the present invention provides a method of treating pain associated with tissue lancing. In one embodiment, the method includes applying a therapeutically effective amount of a topical composition including loperamide or a pharmaceutically acceptable salt thereof to a region of skin of a patient; where the region is a region selected for tissue lancing. In another embodiment, the topical composition further includes between 7% and 3% weight loperamide or a pharmaceutically acceptable salt thereof. In yet another embodiment, the topical composition further includes between 30% and 40% weight propylene glycol, between 30% and 40% ethanol; between 25% and 35% ethyl acetate and between 3% and 0.5% klucel HF. The topical composition may be a cream, gel, ointment, paste, lotion, emulsion, viscous liquid, foam or a semisolid. The pharmaceutically acceptable salt may be loperamide HCl.

The patient may be a human patient, for example, a human adult, child or neonate patient. In one embodiment, the human patient is less than one month old. In another embodiment, the patient is a diabetic patient. The tissue lancing may be a heel lancing or a finger lancing. The tissue lancing may include a plurality of individual lancings and the therapeutically effective amount may be an amount that at least reduces synaptic changes resulting from the tissue lancing.

### BRIEF DESCRIPTION OF THE DRAWINGS

FIG. 1 is a graph illustrating effects of the application of topical loperamide 5% gel on pain following finger lancing measured by VAS score 40 minutes and numeric pain score at 24 hours following L (loperamide) or P (placebo) gel application. The means are represented, independent sample t-test comparison of pain scores, **p<0.01.

FIG. 2 is a graph illustrating effects of topical loperamide application on comparison pain scores between the first lance and second lance delivered in the same area of the fingertip 49 minutes later. Loperamide group (diamond) experienced significantly higher frequency of much less and a little less pain than placebo (square) at lance 2. There were no placebo participants experiencing less pain at lance 2. There was a downward trend toward less pain (solid line) with loperamide and an upward trend of more pain with placebo (dashed line).

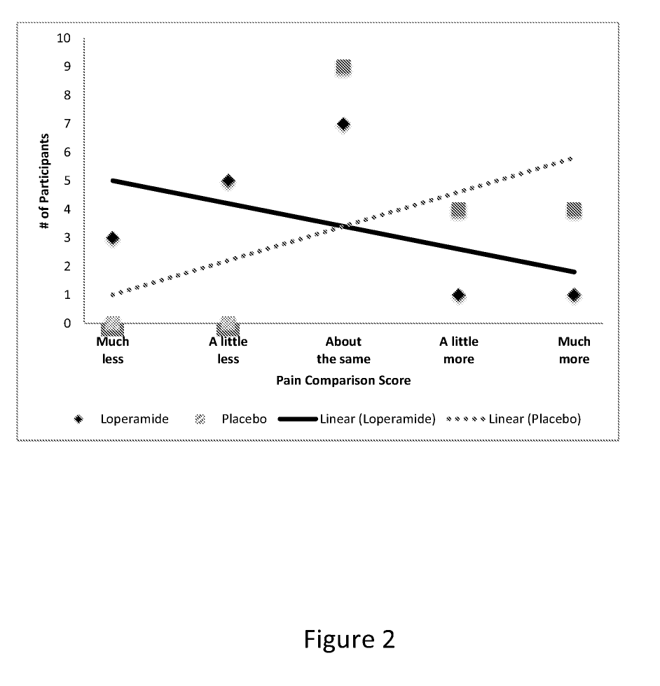

FIG. 3 is a graph illustrating effects of topical loperamide application following 2 finger lancings on the proportion of participants who felt no pain (numeric scale), sensitivity to touch, and sensitivity to pressure at 24 hours. Non-parametric chi-square population percentiles are represented p=<0.005 for all comparisons.

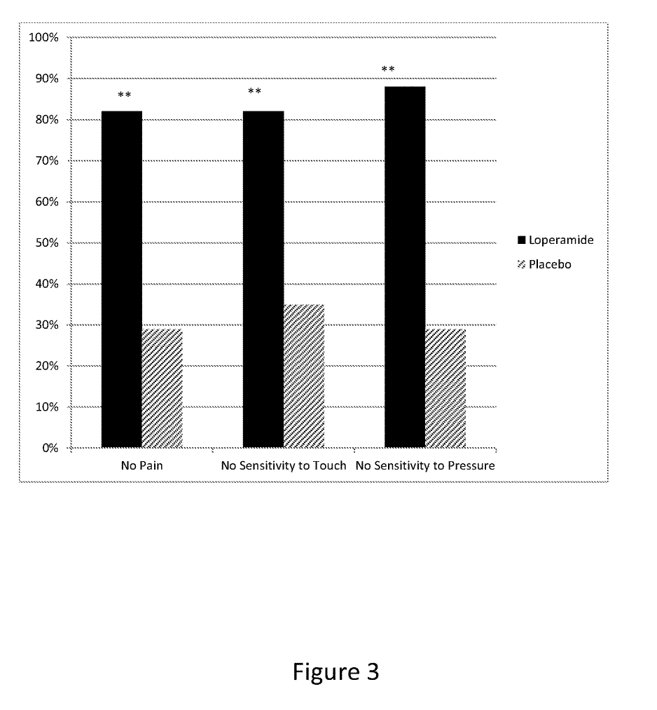

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

The term “therapeutic effect” as used herein means an effect which induces, ameliorates or otherwise causes an improvement in the extent of pain, for example, repeated procedural pain, such as that resulting from finger and heel lancing.

As used herein, the term “patient” refers to a human or veterinary patient. In one embodiment, the veterinary patient is a mammalian patient.

### Topical Loperamide Composition and Method of Use Thereof

Animal studies of inflamed paws in thermal, mechanical and chemical models, revealed that application of the opioids morphine and fentanyl topically reduced pain ostensibly through a local action.^{22-24, 38-40} However, pain responses were also reduced in the contralateral paw suggesting systemic distribution through topical administration. These studies led to human case reports of similar efficacy in patients.²² This raises the possibility that topical opioids could produce local relief from pain if systemic distribution and its associated side effects were markedly reduced. The antidiarrheal loperamide (4-[4-(4-Chlorophenyl)-4-hydroxypiperidin-1-yl]-N,N-dimethyl-2,2-diphenylbutanamide) has significantly reduced CNS effects resulting from its high affinity for p-glycoprotein.³⁵⁻³⁷

Loperamide, a piperdine derivative with a structure similar to the synthetic opioid meperidine, has strong affinity for Mu opioid receptors. It was approved by the FDA in 1969 as an anti-diarrheal agent with Mu opioid activity mimicking the constipating effects of other opioids, but with markedly reduced CNS effects due to its affinity for p-glycoprotein, preventing crossing of the blood brain barrier (BBB) under normal circumstances.³⁵⁻³⁷ Loperamide has a high safety record and has been available for the past 40 years as an FDA regulated over-the counter (OTC) oral, anti-diarrhea safe for use in adults, children and during pregnancy.³⁷

Loperamide was synthesized in the 1960's as an anti-diarrheal agent with direct action on opioid receptors in the gut. It has very limited central bioavailability, and thus is devoid of CNS effects including having no analgesic effects when given systemically. In the late 1980's opioid receptors were identified on peripheral sensory neurons. Opioid receptors extend along the sensory neurons on C- and Aδ-fibers, vanniloid receptor-1-positive visceral fibers and on neurons expressing isolectin B4, substance P and calcitonin-gene-related peptide. Single mu (MOR) delta (DOR) and kappa (KOR) opioid receptors are present with MOR being most abundant. Similar to the CNS, activation of peripheral opioids produces a dose-dependent analgesia. The mechanism of action of peripheral opioid receptors is a reduction in the excitability of peripheral nociceptor terminals and a decrease in the propagation of action potentials. Calcium channels, sodium channels, glutamate receptors, K+ channels and other G-protein coupled receptors (GPCR) are involved in the peripheral mechanism of action. Subsequent work identified that the direct local injection of peripheral opioids in inflamed paws in thermal, mechanical and chemical models had antihyperalgesic properties through a local action. Such studies led to human reports of such efficacy. Topical application of morphine in human case reports in chronic wounds and open wounds report rapid relief with no systemic absorption or adverse effects. Case studies in abrasion, erythema and frostbite identified less hyperalgesia in response to tactile, heat and pressure with loperamide cream.

One aspect of the present invention provides a topical composition including loperamide, a loperamide analog, or a pharmaceutically acceptable salt thereof. The loperamide analog may be an analog as disclosed in U.S. Pat. No. 6,166,036, issued Dec. 26, 2000, or U.S. Pat. No. 5,994,372, issued Nov. 30, 1999, the contents of which patents are hereby incorporated by reference. In certain embodiments, the loperamide, loperamide analogue or pharmaceutically acceptable salt thereof is provided as a topical composition.

Such a composition may be in the form of a hydrophilic gel, in which the polar phase includes at least one water-soluble or water-dispersible hydrophilic solvent other than water. The compositions may optionally also contain water, especially if water is present in one of the solvents, such as Ethanol USP 190 proof. Suitable hydrophilic components include Dehydrated Alcohol USP (Ethanol 200 proof), Alcohol USP (Ethanol 190 proof), Specially Denatured Alcohol, ethyl acetate, one or more glycols such as polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof. Alternatively, the hydrophilic gel can thickened by suitable natural, modified natural or synthetic polymers such as disclosed herein.

The composition may be in the form of a thickened hydroalcoholic gel, including a blend of water and alcohol as the polar phase which has been thickened by suitable natural, modified natural or synthetic polymers. Alternatively, the thickened hydroalcoholic gels can be thickened using suitable polyethoxylate alky chain surfactants or other nonionic, cationic, or anionic systems. The alcohol can be ethanol, isopropyl alcohol or other pharmaceutically acceptable alcohol.

The composition may be in the form of a thickened aqueous gel including an aqueous phase thickened by suitable natural, modified natural or synthetic thickeners. These aqueous gels may also include one or more solvents. Alternatively, the aqueous gels may be thickened using suitable polyethoxylate alky chain surfactants or other nonionic, cationic or anionic systems.

The composition may be in the form of an oil-in-water emulsion including a discrete phase of a hydrophobic component and a continuous aqueous phase that includes water and optionally one or more polar hydrophilic excipients. These composition may also include solvents, co-solvents, salts, surfactants, emulsifiers and other components. In addition, the composition may include water-soluble or water-swellable polymers that help to stabilize the emulsion.

The compositions may be in the form of a water-in-oil emulsion including a continuous phase of a hydrophobic component and an aqueous phase including water and optionally one or more polar hydrophilic carrier(s), as well as salts and other components. The emulsion may also include oil-soluble or oil-swellable polymers as well as one or more emulsifier(s).

The composition may be in the form of a hydrophilic or hydrophobic ointment, formulated with a hydrophobic base (e.g. petrolatum, thickened or gelled water insoluble oils, and the like) and optionally having a minor amount of a water soluble phase. Hydrophilic ointments may contain one or more surfactants or wetting agents.

The composition may include one or more solvents or co-solvents to obtain the desired level of active ingredient solubility in the topical product. The solvent may also modify skin permeation or activity of other excipients contained in the topical product. Solvents include acetone, alcohol (ethanol), benzyl alcohol, butyl alcohol, diethyl sebacate, diethylene glycol monoethyl ether, diisopropyl adipate, dimethyl sulfoxide, ethyl acetate, isopropyl alcohol, isopropyl isostearate, isopropyl myristate, propylene glycol and SD alcohol.

The compositions may include one or more surfactants to emulsify the composition and to help wet the surface of actives or excipients. Such surfactants include, for example, alkyl aryl sodium sulfonate, Amerchol-CAB, ammonium lauryl sulfate, apricot kernel oil Peg-6 esters, Arlacel, benzalkonium chloride, Ceteareth-12, Ceteareth-15 Ceteareth-30, cetearyl alcohol/ceteareth-20, cetearyl ethylhexanoate, Cetheth-10, Ceteth-2, ceteth-20, ceteth-23, choleth-24, cocamide ether sulfate, cocamine oxide, coco betaine, coco diethanolamide, coco monoethanolamide, coco-caprylate/caprate, disodium cocoamphodiacetate, disodium lauryl sulfosuccinate, disodium oleamido monoethanolamine sulfosuccinate, docusate sodium, laureth-2, laureth-23, laureth-4, lauric diethanolamide, lecithin, mehoxy PEG-16, methyl gluceth-10, methyl gluceth-20 m mehyl glucose sesquistearate, oleth-2, oleth-20, PEG 6-32 stearate, PEG-100 stearate, PEG-12 glyceryl laurate, PEG-120 methyl glucose dioleate, PEG-15 cocamine, PEG-150 distearate, PEG-2 stearate, PEG-20 methyl glucose sesqustearate, PEG-22 methyl ether, PEG-25 propylene glycol stearate, PEG-4 dilaurate, PEG-4 laurate, PEG-45/dodecyl glycol copolymer, PEG-5 oleate, PEG-50 Stearate, PEG-54 hydrogenated castor oil, PEG-6 isostearate, PEG-60 hydrogenated castor oil, PEG-7 methyl ether, PEG-75 lanolin, PEG-8 laurate, PEG-8 stearate, Pegoxol 7 stearate, pentaerythritol cocoate, poloxamer 124, poloxamer 181, poloxamer 182, poloxamer 188, poloxamer 237, poloxamer 407 or combinations of a least two of these agents.

For certain applications, it may be desirable to formulate a topical composition that is thickened with soluble, swellable, or insoluble organic polymeric thickeners such as natural and synthetic polymers or inorganic thickeners such as acrylates copolymer, carbomer 1382, carbomer copolymer type B, carbomer homopolymer type A, carbomer homopolymer type B, carbomer homopolymer type C, caroboxy vinyl copolymer, carboxymethylcellulose, carboxypolymethylene, carrageenan, guar gum, hydroxyethyl cellulose, hydroxypropyl cellulose, microcrystalline wax or methylcellulose.

The composition may include additional components conventionally found in cosmetic and pharmaceutical topical products. Such components may include, for example, antifoaming agents, preservatives, antioxidants, sequestering agents, stabilizers, buffers, pH adjusting solutions, skin penetration enhancers, film formers, dyes, pigments, fragrances and other excipients to improve the stability or aesthetics of the topical product.

Other suitable topical compositions are disclosed in U.S. Pat. No. 6,355,657, “System for percutaneous delivery of opioid analgesics”, Issued Mar. 12, 2002, the contents of which are hereby incorporated by reference.

Another aspect provides a method of treating pain associated with tissue lancing or other procedure involving pain to the surface of the body of a patient, for example, percutaneous central line placement or venipuncture. One embodiment includes applying a therapeutically effective amount of the topical composition including loperamide, a loperamide analog or a pharmaceutically acceptable salt thereof to a region of the skin of a patient selected for the tissue lancing or other procedure. The pharmaceutically acceptable salt may be, for example, loperamide HCl.

The composition may include, for example, between 10% and 2%, or 7% and 3%, or 6% and 4%, or about 5% weight loperamide, a loperamide analog or a pharmaceutically acceptable salt thereof. The composition may also include between 20% and 50%, or 30% and 40%, or 33% and 37% weight propylene glycol. In another embodiment, the composition may include between 20% sand 50%, or 30% and 40%, or 33% and 37% ethanol. In yet another embodiment, the composition may include between 20% and 40%, or 25% and 35%, or 27% and 33% ethyl acetate. In another embodiment, the composition may include between 5% and 0.1%, or 4% and 0.3%, or 3% and 0.5% klucel HF. In yet another embodiment, the composition includes between 30% and 40% weight propylene glycol, between 30% and 40% ethanol; between 25% and 35% ethyl acetate and between 3% and 0.5% klucel HF. The composition may be, for example, in the form of a cream, gel, ointment, paste, lotion, emulsion, viscous liquid, foam or a semisolid.

In one embodiment, the patient is a human patient, for example, a human adult, child or neonate patient. In another embodiment, the human patient is less than one month old. In yet another embodiment, human patient is a diabetic patient.

The lancing may be a lancing of any part of the body, for example, a heel lancing or a finger lancing. In certain embodiments, the effects due to pain are reduced then the patient is subjected to a number of lancings over a given time period. In another embodiment, the therapeutically effective amount is an amount that at least reduces synaptic changes at the site of the pain stimulus. In yet another embodiment, the therapeutically effective amount is an amount that activates opioid receptors at the site of the pain stimulus and/or alleviates immediate pain, hyperalgesia and the exaggerated pain state from increased afferent activity. In another embodiment, the therapeutically effective amount is an about that does not result in any or any significant amount of systemic absorption of the loperamide.

One embodiment the present invention provides a method of improving the health of the at-risk neonate by reducing the pain and physiologic response to the commonly repeated lancing or other procedure, for example, heel lancing. Lancing the finger and heel to obtain capillary blood for specimen collection and diagnostic testing is a painful and tissue damaging procedure. The lancet directly splices the capillaries along with free nerve endings resulting in a sharp localized pain response. Furthermore, neonates are particularly vulnerable to synaptic restructuring from repeated manipulation and painful stimuli. The present method provides for the reduction or elimination of such restructuring.

Puncturing the heel with a lancet for blood specimen collection is one of the most frequent procedures in the hospitalized neonate. The neonate responds to the puncture with physiologic distress increased heart rate, blood pressure and cerebral pressure; decreased oxygen saturation, a red inflamed heel and long term conditioned anxiety and exaggerated pain responses. High risk neonates are a significantly understudied population and there is currently no topical agent effective for repeat lancing pain.

The present method may also have application for children and adults, for example, adult or child diabetics, adult or children on anti-coagulants. Such patients may require repeat lancing for glucose monitoring or INR monitoring. The pain associated with lancing has been cited as a reason for non-compliance in the diabetic population.

For example, loperamide, formulated as a solution/cream at a concentration of 5% (5 gm per 100 ml) may be applied topically to the finger-tip or heel and lightly rubbed into the tissue for 1 minute and left in place for 30 minutes to effectively produce analgesia, with the effect remaining at 24 hours. The 5% cream concentration may provide anti-hyperalgesic effectiveness and the ability for skin penetration through the stratum corneum. The compound may be formulated to penetrate the skin with no, or no significant, systemic absorption and to utilize standard carrier compounds which are non-irritating and or non-sensitizing and possess a neutral pH. The topical composition may include: 5% Loperamide Hel; 30% Propylene Glyco/; 34% Ethanol (190 proof USP); 30% Ethyl acetate; Klucel HF 1%. All of the solvents used for the topical cream loperamide composition or cream alone are approved by the FDA as inactive ingredients.

The following examples are provided to enable those of ordinary skill in the art to make and use the methods and compositions of the invention. These examples are not intended to limit the scope of what the inventors regard as their invention. Additional advantages and modifications will be readily apparent to those skilled in the art.

### Example 1—Preparation and Application of Loperamide Composition

Loperamide hydrochloride powder, (Sigma Aldrich) was developed as a topical solution at a concentration of 5% (5 gm per 100 ml). Each 0.2 gm application (equivalent to 10 mg) was applied to the finger-tip and forearm. The topical composition includes 5% Loperamide HCl; 30% Propylene Glycol; 34% Ethanol (190 proof USP); 30% Ethyl acetate; and Klucel HF 1%. The placebo solution contained the same ingredients without the loperamide. All of the compounds in the solution are FDA approved as inactive ingredients safe for use as inactive ingredients.⁴⁶ The 5% gel concentration was chosen due anti-hyperalgesic effectiveness and the ability for skin penetration through the stratum corneum. The materials were compounded in a pharmacy that meet rigorous microbial control standards and diligently follow USP <795> guidelines.⁴⁷ Stability using the Loperamide Hydrochloride USP Assay method (titration) was used to establish that the drug product remained within specification for loperamide hydrochloride content for at least six months following compounding.

### Example 2—Participants and Randomization of Study

A convenience sample of 34 healthy adult volunteers were evaluated for the effects of loperamide gel (L) or gel alone which was designated as the placebo (P). Pain levels were assessed following 2 fingersticks. Participants who met the inclusion criteria were randomized to either receive 5% loperamide gel or gel alone. Individuals were excluded from participating if they had a history of drug induced hypersensitivity reactions; took any anti-inflammatory medications in the past 12 hours; if they routinely performed finger lancing for blood specimen monitoring (e.g. diabetes); or had calloused fingers pads. The study was completed in a health center office under the oversight of a medical doctor.

Randomization was completed using a random number generator which dictated the treatment assignment into either the loperamide group (n=17) or the gel alone group (n=17). The solutions looked and smelled identical and the sample amounts were drawn up into a syringe based on the random number generator and placed into separate envelopes labeled participant 1 to participant 34. The researcher doing the lance and applying the solution was blinded to the assignment. In case of an emergency, an email could be opened by the researcher listing the participant number and assignment (loperamide or placebo), with the sender notified that the email was opened. There were no emergencies during this study.

### Example 3—Study Design

Peripherally applied loperamide, similar to the effects of morphine and fentanyl, produced a rapid dose-dependent antinociception in rats following thermal, chemical or abrasion-like injury to the hind paw.³⁸⁻⁴⁴ Topical loperamide 5% cream lightly rubbed on the paw for one minute and then left in place for 10 minutes, produced significant analgesia as well when applied both before and after injury.⁴⁴ However, animal studies revealed systemic effects of morphine and fentanyl following topical application, whereas loperamide, as a substrate for p-glycoprotein revealed none.³⁸⁻⁴⁴ Furthermore, topical C14 30 mg loperamide cream results in a significant uptake in the epidermis and dermis with a peak concentration at 30 minutes but with no detectable radioactivity in serum samples.⁴⁴

The prospective double-blind randomized study to determine the analgesic efficacy and safety of topical loperamide solution in reducing pain during repeat finger lancing in healthy adults was approved by the Institutional Review Board of Rush University Medical Center according to the Declaration of Helsinki, approved by the Food and Drug Administration (FDA) for Investigational New Drug status (IND 122919) and registered with Clinical Trials.Gov NCT 02711891. All volunteers were evaluated by the same nurse with 30 years of heel and finger lancing experience, following strict universal safety precautions and the World Health Organization (WHO) guidelines on drawing blood: best practices in phlebotomy.⁴⁵ All participants signed informed consents and those who completed both sessions of the study received a $20.00 gift card for their time and discomfort.

The 5^{th} digit finger pad of the non-dominant hand was cleaned with a 70% alcohol wipe. The participant was asked to look away and the outside of the finger pad was punctured using a single-use lancet (Surgilance™ Medipurpose, Georgia, USA), which contains a stainless steel blade automatic trigger and retractor that incises with a 21 Ga needle to a controlled depth of 2.2 mm and width of 0.8 mm. A sample of 0.3 ml of blood was collected with each lance by occasional squeezing and milking the finger downward toward the tip, replicating the same technique and minimum volume for specimen collection with newborn heel lancing. Participants rated their pain using a visual analog scale (VAS) within one-minute of lance completion. Ten minutes following lance one, 0.2 grams of loperamide hydrochloride 5% gel or gel alone was applied to the lance site within an 8 mm diameter circular plastic mold. The gel was rubbed lightly on the skin surface for one minute using a gloved finger swirled into the mold site. The mold was removed and the site was covered with an occlusive dressing left in place for 30 minutes. At 30 minutes the gel was removed with gauze, the skin cleaned with an alcohol wipe and the second lance was done within the same location as the first.

During each lance procedure, HR was monitored using a pulse oximeter (Kenek Edge, Vancouver CA) placed on the dominant hand index finger 30 seconds pre, during, immediate post and 30 seconds post lance, with the mean for each phase used as the comparison value. Pain ratings were obtained using a 0-10 (100 mm) VAS and the pain comparison scale was used following lance two. A 24 hour post lancing follow-up text/call was sent to obtain pain (0-10 numeric scale), sensitivity and pressure ratings using questions adapted from the Pain Quality Assessment Scale. We expected to demonstrate that the application of loperamide gel significantly reduced the pain intensity relative to control and gel only.

The safety of loperamide solution was determined by assessment of local skin reactions and stool pattern. Loperamide gel or the gel itself was applied in a thin layer to the forearm skin using the mold and left in place for 30 minutes. The solution was removed with gauze and the skin was immediately assessed for erythema using an erythema scale. The skin was similarly assessed after 24 hours along with any reported changes in stool patterns or abdominal cramps to reflect potential systemic affects from Mu opioid anti-diarrheal action on the gut.

Demographic data included: age, sex, analgesic use, and the anxiety about needle sticks scale with a range from 0 (0=not at all anxious) to 5 (high severity of anxiety). The primary outcome was a reduction in the VAS and pain comparison scale⁴⁸⁻⁵² between the loperamide gel and placebo gel. Participants marked their level of pain the horizontal VAS scale within one minute of each lance. The pain comparison scale included ratings of: much less, a little less, about the same, a little more or much more. Pain and sensitivity at 24 hours was evaluated using a numeric 0-10 pain scale and sensitivity and tenderness questions adapted from the Pain Quality Assessment Scale©. The sensitivity question requested: please tell us how sensitive your fingerstick site has been to light touch or clothing rubbing against it over the past day (0=non sensitive and 1=sensitive). The tenderness questions requested: please tell us how tender your fingerstick pain is when something has pressed against it in the past day. (0=not tender and 1 tender “like a bruise”). Heart Rate was measured using the Kenek Edge Pulse Oximeter System which used a flipclop sensor and companion app that works with an iOS mobile device to measure blood oxygen saturation (SpO₂) and heart rate. The non-invasive, pulse oximeter measures arterial blood oxygen saturation (SpO₂) and pulse rate based on the amount of transmitted, reflected and scattered light through the finger. The HR display range is 30-250 bpm with an accuracy of +/−2 bpm. A standard erythema scale (0=no effect; 0.5=observer indecisive; 1.0=faint pink-no border; 1.5=faint pink border; 2.0=faint pink with one border; 2.5=faint pink with two borders; 3.0=red; 3.5=fiery red; 4.0=violaceous red) was used to assess any hypersensitivity reaction at 30 minutes and 24 hours after gel application. Evaluation for systemic effects included any changes in stool patterns or abdominal cramps identifying any potential systemic affects from Mu opioid's anti-diarrheal action on the gut.

### Example 4—Statistics

An a priori power analysis was determined based on previous adult venipuncture studies using a mean difference of VAS scores of 1.5 cm between groups and a standard deviation of 1.5. A group size of 16 patients per study arm (total study population of 32) was suggested to achieve 80% power (β=0.10) in detecting a two-sided level of significance of 5% (α=0.05).⁵³ A sample size of 34 was used for this study to account for a 10% drop out or incomplete data. Frequency distributions on demographic variables were tabulated. The statistical analysis was performed using SPSS ver. 18 (SPSS Inc. Chicago Ill., USA). An independent sample t-test was used to determine whether the means of the primary endpoints (VAS score and pain comparison score) were different between the groups. A paired sample T-test was used to compare lance one to lance two VAS scores. Heart rate was compared over time using repeated measures ANOVA to determine if the mean HR at each point for both groups was equal. A chi-square was used to evaluate the population proportions of 24 hour pain, sensitivity to touch and sensitivity to pressure. A P value of <0.05 is considered significant for all tests.

### Example 5—Study Results

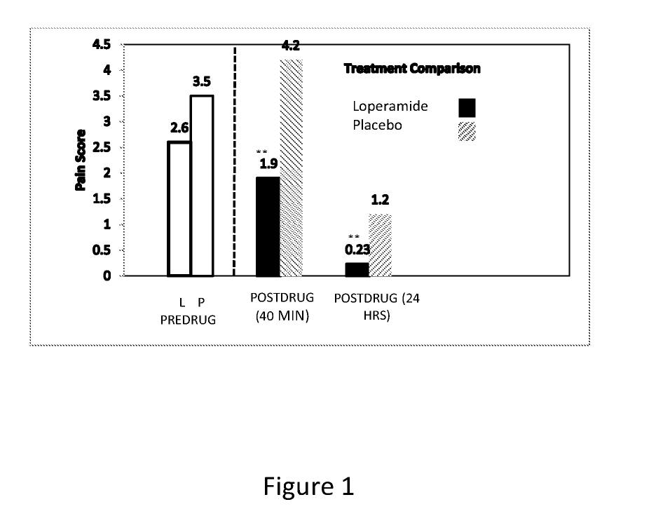

All participants completed the study. The study groups were comparable and though there was a trend toward higher anxiety scores with blood drawing and higher lance one pain ratings in the placebo group, these were not significant between groups. Loperamide 5% gel produced analgesia in the fingertip after 30 minutes of administration. Basal lance one VAS scores were comparable between groups at lance one (L 2.6+/−1.7; P 2.5+/−2.3 p=0.222). After lance one and thirty minutes following loperamide gel or placebo gel application, the loperamide gel reduced the VAS score by 55%. This difference was statistically significant with the VAS scores for loperamide (1.9+/−1.4) and the VAS score for the placebo (4.2+/−2.4) t₍₃₂₎=3.3, p=0.002 FIG. 1). A paired samples t-test was also conducted to compare the within group lance response. There was a significant increase in the VAS scores for lance one placebo group (3.5+/−2.2) and lance two placebo gel (4.2+/−2.4), (t₍₁₆₎=−3.4, p=0.003) suggesting that the pain during the second lance was greater. No differences were observed loperamide group lance one (2.6+/−1.7) and lance two (1.9+/−1.4), (t₍₁₆₎=1.8, p=0.08). There was a significant difference in the mean comparative pain score for the loperamide group (2.5+/−1.0) and the placebo group (3.7+/−0.84), (t₍₃₂₎=3.5, p=0.001). Descriptive cross-tabulations identified that there were no participants in the placebo group rating their pain following the second lance as a little less (0%) or much less (0%) in comparison to the loperamide group where there were 3/17 (17%) rating the pain as much less and 5/17(and 30%) rating the pain as a little less (Table 3).

The proportion of participants who identified having less pain with the second lance was greater for the loperamide group ( 8/17, 47%) in comparison to the placebo group (0%) and the proportion of patients who had a comparison score of more pain or much more pain was greater in the placebo group ( 8/17, 47%) in comparison to the loperamide group ( 2/17, 11%). There was no difference in HR scores between groups at any time in the lancing segment (pre, during lance, during collection and post lance). Heart rate scores during lance one were L 74.6+/−12; P 74.1+/−9.4 p=0.887 and during lance 2 were L 74.8+/−11.4; P 71.8+/−7.1 p=0.457. There were also no significant difference in HR changes within the L group during any phases of the lancing (pre, lance, collect and post).

Loperamide use resulted in significantly less pain and significant less sensitivity to touch and pressure (anti-allodynia) at 24 hours. Overall 24 hour fingertip pain was significantly lower in the loperamide group (independent sample t-test (L 0.23+/−0.56; P 1.23+/−1.4, F=7.13 p<0.01). The 24-hour pain and sensitivity scores were analyzed by a non-parametric chi-square for population proportions. The proportion of participants who felt no pain was greater in the loperamide (82%) vs the placebo (29%) group, X² (1, n=34)=−0.533, p=0.001. The proportion of participants who stated no sensitivity to touch was greater in the loperamide (82.4%) vs. placebo (35%) group, X² (1, n=34)=−0.478, p=0.004. The proportion of participants who felt no sensitivity to pressure was greater in the loperamide (88.2%) vs the placebo (29.4%) group, X² (1, n=34)=−0.598, p=<0.0001, Table 4). Thus the loperamide gel resulted in a significant decrease in pain and sensitivity at 24 hours in comparison to the placebo group for all measures.

Loperamide or placebo gel did not produce any forearm redness at any point (immediate, 30 minutes, or at 24 hours). One participant showed redness around the application site on the forearm in response to the topical adhesive bandage. There were no cramps reported in the loperamide or placebo group and there was no difference between groups for any change in bowel patterns at 24 hours post solution application.

### Example 5—Discussion

The use of loperamide 5% gel, applied topically in healthy adults in a repeat finger lancing model, was effective at reducing the pain and sensitivity to pressure and touch at 24 hours post lancing. There were no hypersensitivity reactions to the loperamide gel on the fingertip or forearm and no evidence of any systemic absorption. There were no differences in HR, which was anticipated since in adults, unlike infants and children, HR is an inconsistent measure of pain due to the magnitude of change equally present with fear or emotions.

Our results indicate that loperamide, presumably acting via a peripheral mechanism, is an effective antinociceptive agent (blocking the sensation of painful stimulus by sensory neurons) in adult volunteers. Building on the rodent and primate studies, the loperamide 5% gel concentration was chosen due anti-hyperalgesic effectiveness and the ability for skin penetration through the stratum corneum. The compound for this study penetrated the skin with no evidence of systemic absorption. However systemic absorption cannot be ruled out since alterations in bowel activity, a well-known effect of opioids as well as loperamide, is a crude index of systemic effects. Indeed short-term clinical studies with topical fentanyl, known to be absorbed systemically, similarly revealed no changed in bowel function. Further studies assessing loperamide in plasma are therefore needed to access the potential for systemic effects.

Limiting factors for topical analgesics in human studies have centered on a concentration and a composition that can penetrate through the stratum corneum and deliver the active ingredient to the topical nerves in sufficient quantity for a sufficient duration of time. The composition for this study was based upon U.S. Pat. No. 6,355,657 B1⁵⁴, the contents of which are hereby incorporated by reference. The solvent mixture was tested using skin permeation techniques with human abdominal cadaver skin. The “657” patent defined the scope of a composition with loperamide at least 1% concentration having an in vitro rate of penetration through the stratum corneum which deposits the drug into the stratum corneum and allows the drug to diffuse at a flux rate of at least about 0.3 μg/cm²/hour. The mixed solvent provided twice the skin permeability delivery than the ADL-2-1294-B cream originally developed by Yaksh.⁵⁵ Based on the 5% concentration which is 5 gm per 100 ml applied to an 8 mm diameter mold, an application of 0.2 cc or 0.2 gms is equivalent to 10 mg or 10,000 μg. The 5% concentration could result in absorption increase from 0.3 to 1.5 μg per cm2 per hour or 0.015 mg, which is far below the standard adult oral dose of 2 mg or daily dose of 16 mg. Considering the bi-phasic half-life with a rapid phase one of 2.3 hours and a slow phase two of 23 hours,⁴⁰ even if the loperamide continued to be absorbed across the skin over a 24 hour period, the maximum dose that could be absorbed during that time is 0.36 mg (1.5 μg×24 hours), which is far below the adult maximum oral dose of 16 mg in 24 hours. Thus, topically applied, loperamide gel used in this study would be expected to produce minimal systemic effects even if it was found to distribute body wide. Moreover, as a p-glycoprotein substrate, its entry into the brain would be virtually nil overcoming the potential CNS effects of morphine, and especially fentanyl, which is known to produce CNS toxicity when delivered via a patch.⁵⁶

In previous animal studies, subcutaneous delivery of morphine or fentanyl to produce localized pain relief, affected both the inflamed and non-inflamed paw.³⁸⁻⁴⁴ This suggests that these agents affected the contralateral paw either through systemic distribution to the contralateral aw or into the brain. Loperamide only affected the inflamed paw. In addition, the effects seen with morphine and fentanyl were reversed by naltrexone but not methylnaltrexone, whereas the anti-allodynic effect of loperamide was sensitive to both.³⁸⁻⁴⁰ Naltrexone occupies both central and peripheral Mu opioid receptors, whereas methylnaltrexone does not penetrate the BBB. Taken together, these data suggest that the contralateral effects of morphine and fentanyl were a consequence of CNS action whereas loperamide acts only peripherally.

Another safety consideration in this model is the inflammatory source. A lancet is being introduced and loperamide is being applied to a site where the stratum corneum has been damaged and which could allow for increased penetration of the drug. In models specifically designed to break the stratum corneum with an array of micro-needles for the purpose of enhancing drug delivery, there is a 3.8 fold increase in drug delivery.⁵⁷ Assuming the enhanced absorption could apply to this model, then there could be an increased penetration from 0.015 mg to 0.057 mg of loperamide, which is still significantly below the 2 mg starting oral dose.

The gel utilized approved FDA inactive ingredients in a standard composition known to be non-irritating and/or non-sensitizing and possess a neutral pH, which was borne out in this present study where no redness was observed at any site of application. While the efficacy of topical loperamide an anti-hyperalgesic has been established in animal models, there have been no prior clinical studies completed with loperamide gel besides an oral ulcer mouth rinse case report by Nozaki-Taguchi⁵⁸ and other case studies identified in U.S. Pat. No. 6,355,657 B1. Thus, the present study is the first to demonstrate analgesic efficacy following topical application in a prospective double-blind clinical study.

While our results are promising and they reach clinically significant results for the pain comparison scale and 24 hour pain score, they do not reach clinical significance for adult repeat measure VAS scores. The clinically significant difference in VAS scores is reported as 0.5 for single subject pain scores 5 minutes or less apart and 1.3 cm for pain scores 20-30 minutes apart.⁴⁹⁻⁵¹ Results of studies among children with acute pain suggest the minimal clinically significant difference in pain scores to be 1 on a VAS of 1-10.⁵² The results from this study were a mean score decrease for loperamide of 0.8 (1^{St} lance 2.7, 2^{nd} lance 1.9) and an increase in VAS scores for placebo of 0.7 (1^{st} lance 3.5 and 2^{nd} lance 4.2). Additional studies which allow longer penetration time or using higher doses may be needed.

This study also confirms the high level of pain felt during a single lance. The 1^{st} lance pain rating (VAS) was 3.3 with a range of 0-8 (SD 2.5). The comments by 80% of participants included how much the lance hurt. In response to the pain and subsequent non-compliance with finger lancing experienced by adult diabetic patients, there has been extensive technology improvement with new lancets penetrating only to 0.3 mm, (versus the 2.2 mm depth for this study) and quick skin vacuum devises which do not splice the skin at all. While lancing is the most common invasive procedure of the high risk newborn and the guidelines of both the American Academy of Pediatrics and Canadian Paediatric Society identify that prevention of pain in neonates is the goal due to the potential for deleterious consequences, the currently prescribed treatment for infant heel lancing is oral glucose/sucrose.⁵⁹ The pooled results from over 21 studies utilizing glucose/sucrose identify an overall lower pain score with glucose/sucrose, but no significant difference in HR, oxygen saturation or respiratory rate and no significant change in the cry behavior during the actual lance.⁶⁰ Thus it has been inferred that sucking on a pacifier with sucrose serves to distract the infant and supports coping rather than an actual decrease in pain. Numerous other topical agents have been tried, such as 4% amethocaine (tetracaine) gel, lignocaine ointment, liposomal lidocaine (4% cream) and topical lidocaine-prilocaine (EMLA), but while effective analgesics for skin lancing, line insertions and minor procedures in adults and children over 1 year, in the infant less than one and premature infant population, there is no or minimal effect.¹⁴⁻¹⁹

The safety of any topical agent used in newborns requires close monitoring due to the potential for increased percutaneous absorption of topically applied agents and reduced enzyme activity affecting drug metabolism. Loperamide was specifically chosen due to the historical safety record with children, its limited ability to cross the BBB and the results from all animal and adult case studies that support subcutaneous absorption only. While oral loperamide (Imodium®, McNeil PPC.) was previously approved for use with infants as an oral antidiarrheal agent, a recent meta-analysis reported increased adverse events in children who are younger than 3 years, malnourished, moderately or severely dehydrated, systemically ill, or have bloody diarrhea and that the adverse events (sedation and ileus) outweigh benefits even at oral doses 0.25 mg/kg/d.⁶¹ While the topical loperamide gel composition did not show any evidence of systemic absorption, the efficacy and safety with repeated use in both adults and children would have to be studied before application in the infant population. While the long-term goal of this study is loperamide gel use in infants, consideration to alleviate the pain from repeat lancing for glucose monitoring in the short-term would be to use the adult low penetration lancets where there is less penetration through free nerve endings.

Overall, this present study shows that loperamide, acting as a peripherally selective Mu opioid agonist can act as an antinociceptive agent and prevents the allodynia that occurs with repeat finger lancing. The study also supports the safety of the gel composition with no participants reporting any skin redness or itching and no reports of any signs of systemic absorption.

TABLE 1
Response to Peripheral Opioid Receptor Activation

| Channel/receptor | Response to peripheral opioids |
|---|---|
| Calcium channels | Opioids inhibit Gi protein coupling, resulting in decreased |
| | propagation of action potentials. |
| Terodotoxin- | Opioids inhibit adenylate cyclase resulting in decreased |
| resistant voltage | spontaneous ectopic impulse generation at the injury site. |
| gated sodium | Found only in chronic pain states. |
| channels | |
| Glutamate receptors | Opioids suppress glutamate evoked nociception on A and C |
| | fibers. Inhibit N-methyl-d-aspartate (NMDA) alpha-amino- |
| | 3-hydroxy-5-methyl-4-isoxazolepropionic acid (AMPA) & |
| | kainite (KA) localized on unmyelinated and myelinated |
| | sensory axons in the skin. |
| Vanilloid VR1 | Opioids inhibit the triggering vanilloid VR1 receptors, which |
| receptors | are up-regulated with inflammation. |
| GIRK channels | Opioids stimulate GPCR's interacting with GIRK channels |
| | to open and become more permeable to inward K+, resulting |
| | in hyperpolarization. |
| Pro-inflammatory | Opioids reduce substance P, calcitonin-gene-related peptide, |
| peptides | and cytokine release from peripheral sensory nerve endings |
| | resulting in reduced edema, vasodilation and extravasation |

TABLE 2
Study Population Demographic Variables

| Demographic | Range | Mean | SD |
|---|---|---|---|
| Age | 18-72 years | M 41.5 | (SD 15.9) |
| Anxiety with blood | 0 to 8 | M 2.9 | (SD 2.5) |
| draw | | | |
| Gender | Men 10 | | |
| | Women 24 | | |

TABLE 3
t-test Results Comparing Loperamide and Placebo

| | Loperamide | | Placebo | | | |
|---|---|---|---|---|---|---|
| | n = 17 | | N = 17 | | | |
| | 5 male/12 female | | 5 male/12 female | | | |
| | M | SD | M | SD | t-test | Sig. |
| Anxiety | 2.8 | 2.6 | 3.2 | 2.6 | .467 | .644 |
| Blood Draw | | | | | | |
| VAS Lance 1 | 2.6 | 1.7 | 3.5 | 2.2 | 1.24 | .222 |
| VAS Lance 2 | 1.9 | 1.4 | 4.2 | 2.4 | 3.33 | .002 |
| Comparison | 2.5 | 1.0 | 3.7 | .84 | 3.55 | .001 |
| Pain Score | | | | | | |
| VAS 24 hrs. | .23 | .56 | 1.2 | 1.4 | 2.67 | .012 |

### REFERENCES

1. Centers for Disease Control and Prevention. National Diabetes Statistics Report: Estimates of Diabetes and Its Burden in the United States, 2014. Atlanta, Ga.: US Department of Health and Human Services; 2014
2. Arena J E. Skin to calcaneus distance in the neonate. Archieves of Disability in Children Fetal Neonatal Education. 2005; 990:F32.
3. Fruhstorfer H. Capillary blood volume and pain intensity depend on lancet penetration. Diabetes Care. 2000; 23:562-3.
4. Owens M, Todt E. Pain in infancy: neonatal reaction to a heel lance. Pain. 1984; 77-86.
5. Baker D. Exposure to invasive procedures in neonatal intensive care units admissions. Archives of Disabled Children Fetal Neonatal Education. 1995; F47-F48.
6. Vertanen H. An automatic incision device for obtaining blood samples from the heels of preterm infants causes less damage than a conventional manual lancet. Archives of Disability in Child Fetal Neonatal Education. 2001; 84:F53.
7. Johnson C C. Acute pain response in infants: a multidimenional description. Pain. 1986; 24:373-382.
8. Slater L, Asmerom Y, Boskovic D S. Procedural pain and oxidative stress in premature neonates. The Journal of Pain. 2012; 13 (6):590-597.
9. Taddio A, Shah V, Glibert-MacLeod C, Katz J. Conditioning and hyperalgesia in neweborns exposed to repeated heel lances. JAMA. 2002; 288:857-861.
10. Taddio A K. The effects of early pain experience in neonates on pain experiences in early infancy and childhood. Pediatric Drugs. 2005; 245-47.
11. Grunau R E. Neonatal pain in very preterm infants: long-term effects on brain neurodevelopment and pain reactivity. Rambam Malmonides Medical Journal. 2013; 4:1-13.
12. Page G. Are there long term consequences of pain in newborn or very young infants? Journal of Perinatal Education. 2004; 13:10-17.
13. Anand K J, Scalzo F M. Can adverse neonatal experiences alter brain development and subsequent behavior? Biology of the Neonate. 2000; 77:68-82.
14. Evans J, McCartney E, Lawhon J. Longitudinal comparison of perterm pain responses to repeated heelsticks. Pediatric Nursing. 2005:31:216-21.
15. Shlomowitz, A., & Feher, M. D. (2014). Anxiety Associated With Self Monitoring of Capillary Blood Glucose. British Journal of Diabetes and Vascular Disease, 60-63.
16. Lemyre B, Sherlock R, Hogan D. How effective is tetracaine 4% gel before a peripherally inserted central catheter, in reducing procedural pain in infants: a randomized double-blind placebo controlled trial. BMC Medicine. 20064:11.
17. Larsson B A, Jylli L, Lagercrantz H, Olsson G L. Does a local anaesthetic cream (EMLA) alleviate pain from heel lancing in neonates? ACTA Anaesthesiol Scand. 1995; 39:1028-31.
18. Stevens B, Johnston C, Taddio A. Management of pain from heel lance with lidocaine-prilocaine (EMLA) cream: is it safe and efficacious in preterm infants? J Dev Behav Pediatr. 1999; 20:216-21.
19. O'Brien L, Taddio A, Lyszkiewicz D. A critical review of the topical local anesthetic amethocaine (Ametop) for pediatric pain. Pediatric Drugs. 2005; 7:41-54.
20. Taddio A, Soin H. Liposomel lidocaine to improve procedural success rates and reduce procedural pain among children: a randomized controlled trial. Canadian Medical Association Journal. 2005; 172:1691-95.
21. Taddio A, Lee C, Yip A. Intravenous morphine and topical tetracaine for treatment of pain in preterm neonates undergoing central line placement. JAMA. 2006; 295:793-800.
22. Stein C, Schafer M, Machelska H. Attacking pain at its source new perspectives on opioids. Nature Medicine. 2003; 9:1003-08.
23. Stein C. The control of pain in peripheral tissue by opioids. Mechanism of Disease. 1995; 332:1685-1690.
24. Stein C. Non-analgesic effects of opioids: peripheral opioid effects on inflammation and wound healing. Current Pharmaceutical Design. 2012; 18: 6053-69.
25. Stein C, Hassan A, Przewlocki R. Opioids from immunocytes interact with receptors on sensory nerves to inhibit nociception in inflammation. Proceedings of the National Academy of Science. 1990; 87:5935-39.
26. Borgland S C, Christie M. Nociception inhibits calcium channel currents in a subpopulation of small nociceptive trigeminal ganglion neurons in mouse. Journal of Physiology. 2001; 536:35-47.
27. Schroeder J, Fischback P, Zheng D. Activation of opioid receptors inhibits transient high and low threshold Ca2++ currents but spares a sustained current. Neuron. 1991; 6:13-20.
28. Caterina M, Julius D. The vanilloid receptor: a molecular gateway to the pain pathway. Annual Review of Neuroscience. 2001; 24:487-512.
29. Nockemann D, Rouault M, Labuz D. The K+ channel GIRK2 is both necessary and sufficient for peripheral opioid-mediated analgesia. EMBO Molecular Medicine. 2013; 5:1263-1277.
30. Mousa S. Morphological correlates of immune mediated peripheral opioid analgesia. Advances in Experimental Medicine & Biology. 2003; 521:77-87.
31. Mousa S A, Zhang Q, Sitte N. Endorphin containing memory cells and Mu opioid receptors undergo transport to peripheral inflamed tissue. Journal of Neuroimmunology. 2001; 115:71-8.
32. Hagiwara K, Nakagawasai O, Murata A. Analgesic action of loperamide, an opioid agonist and its blocking action of voltage-dependent Ca++ channels. Neuroscience Research. 2003; 46:493-97.
33. Nockemann D, Rouault M, Labuz D. The K+ channel GIRK2 is both necessary and sufficient for peripheral opioid-mediated analgesia. EMBO Molecular Medicine. 2013; 5:1263-1277.
34. Janson W, Stein C. Peripheral opioid analgesia. Current Pharmaceutical Biotechnology. 2003; 270-4.
35. Schinkel A. P-glycoprotein, a gatekeeper in the blood-brain barrier. Adv Drug Delivery Review. 1999; 36:179-94.
36. Shriver D. Loperamide. In: Goldberg M, ed. Pharmacologic and Biochemical Properties of Drug Substances Washington D.C.: American Pharmaceutical Association Press. 1981: 467-76.
37. FDA. FDA approved drug products: Loperamide hydrochloride. US Department of Health and Human Services. 2014. http://www.accessdata.fda.gov/scripts/cder/drugsatfda/index.cfm?fuseaction=Search.Overview&DrugName=LOPERAMIDE%20HYDROCHLORIDE. Accessed March 2014
38. DeHaven-Hudkins D, Cowan A. Loperamide (ADL2-1294), an opioid antihyperalgesic agent with peripheral selectivity. Journal of Pharmacology and Experimental Therapeutics. 1999; 494-502.
39. DeHaven-Hudkins D, Cowan A. Antipueretic and antihyperalgesic actions of loperamide and its analogues. Live Sciences. 2002; 2787-93.
40. DeHaven-Hudkins D, Dolle R. Peripherally restricted opioid agonists as novel analgesic agents. Current Pharmaceutical Design. 2004; 10(7):743-57.
41. Butelman E, Harris T, Kreek M. Antiallodynic effects of loperamide and fentanyl against topical capsaicin-induced allodynia in anaesthetized primates. Journal of Pharmacology & Experimental Therapeutics. 2004; 311(1):155-63.
42. Coggershall R E, Xhou S, Carlton S M. Opiate receptors on peripheral sensory axons. Brain Research. 1997; 126-32.
43. Menendez L, Lastra A, Meana A. Analgesic effects of loperamide in bone cancer pain in mice. Pharmacology Biochemistry and Behavior. 2005; 81(1):114-21.
44. Nozaki-Taguchi, N, Yaksh T. Characterization of the antihyperalgesia action of a novel peripheral mu-opioid receptor agonist-loperamide. Anesthesiology. 1999; 90(1):225-34.
45. WHO. WHO Guidelines on Drawing Blood. World Health Organization. 2010. http://whqlibdoc.who.int/publications/2010/9789241599221_eng.pdf Accessed Apr. 1, 2013.
46. FDA. Inactive Ingredient Search for Approved Drug Products. US Food and Drug Administrative Services, 2013, Sep. 16. http://www.accessdata.fda.gov/scripts/cder/iig/getiigWEB.cfm Accessed March 2014
47. United States Pharmacopeial Convention. <795? Pharmaceutical Compounding—Nonsterile Preparations. Revised Bulleting Jan. 1, 2014. http://www.usp.org/sites/default/files/usp_pdf/EN/gc795.pdf. Accessed March 2014.
48. Scott J, Huskisso E. Graphic representation of pain. Pain. 1976; 2(2):175-84
49. Bird S, Dickson E. Clinically significant changes in pain along the visual analog scale. Annals of Emergency Medicine. 2001; 639-43.
50. Powell C K. Determining the minimum clinically significant difference in visual analageu pain score for children. Annals of Emergency Medicine. 2001; 37(1):28-31.
51. Yamamoto L, Nomura J, Sato R. Minimum clinically significant VAS differences for simultaneous (paired) interval serial pain assessments. American Journal of Emergency Medicine. 2003; 170-79.
52. Todd K, Funk K. Clinical significance of reported changes in pain severity. Annals of Emergency Medicine. 1996; 485-89.
53. HyLown Consulting (2015) Power and Sample Size Calculator. Retrieved May 2015. http://powerandsamplesize.com/Calculators/Compare-k-Means/1-Way-ANOVA-Pairwise
54. Osborne D W. (2002). U.S. Pat. No. 6,355,657, the contents of which patents are hereby incorporated by reference
55. Yaksh T, Maycock A. (2003) U.S. Pat. No. 6,573,282, the contents of which patents are hereby incorporated by reference
56. Jumbelic M I. Deaths with transdermal fentanyl patches. American Journal of Forensic Medical Pathology. 2010; 31 (1) 18-21.
57. Kochhar J S, Goh W J, Chan S Y, Kang L. A simple method of microneedle array fabrication for transdermal drug delivery. Drug Development and Industrial Pharmacy. 2013; 39:299-309.
58. Nozaki-Taguchi N, Shutoh M, Shimoyama N. Potential utility of peripherally applied loperamide in oral chronic graft-versus-host disease related pain. Japanese Journal of Clinical Oncology. 2008; 38(1): 857.
59. Barrington K H, Batton D G, Finley G A, Wallman C. Pain in the neonate: an update. A joint statement of the Canadian Paediatric Society Fetus and Newborn Committee and the American Academy of Pediatrics. 2015. Pediatrics 126 (2); 404. Available online at: http://pediatrics.aappublications.org/content/126/2/404.full.pdf÷html
60. Stevens B, Yamada J, Lee G Y, Ohlsson A. Sucrose for analgesia in newborn infants undergoing painful procedures. Cochrane Database Syst Rev. 2013; 31:1465-1858.
61. Su-Ting T L, Grossman D C, Cummings P. Loperamide therapy for acute diarrhea in children: a meta analysis. PLOS Medicine. 2007; 10:1371.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

I claim:

1. A method of treating pain associated with tissue lancing, comprising: applying a therapeutically effective amount of a topical composition comprising loperamide or a pharmaceutically acceptable salt thereof to a region of skin of a patient; wherein the region is a region selected for tissue lancing.

2. The method of claim 1, wherein the topical composition comprises between 7% and 3% weight loperamide or a pharmaceutically acceptable salt thereof.

3. The method of claim 2, wherein the topical composition comprises between 6% and 5% weight loperamide or a pharmaceutically acceptable salt thereof.

4. The method of claim 1, wherein the topical composition comprises about 5% weight loperamide or a pharmaceutically acceptable salt thereof.

5. The method of claim 1, wherein the topical composition further comprises between 30% and 40% weight propylene glycol, between 30% and 40% ethanol; between 25% and 35% ethyl acetate and between 3% and 0.5% klucel HF.

6. The method of claim 1, wherein the patient is a human patient.

7. The method of claim 6, wherein the human patient is less than one month old.

8. The method of claim 6, wherein the human patient is a diabetic.

9. The method of claim 7, wherein the tissue lancing is a heel lancing.

10. The method of claim 6, wherein the tissue lancing is a finger lancing.

11. The method of claim 1, where the topical composition if a composition selected from the group consisting of cream, gel, ointment, paste, lotion, emulsion, viscous liquid, foam, and a semisolid.

12. The method of claim 1, wherein the topical composition comprises loperamide HCl.

13. The method of claim 1, wherein the tissue lancing comprises a plurality of individual lancings and wherein the therapeutically effective amount is an amount that at least reduces synaptic changes resulting from the tissue lancing.

14. The method of claim 13, wherein the patient is a neonate patient.

15. The method of claim 14, wherein the individual lancings are heel or finger lancings.

16. The method of claim 1, wherein the therapeutically effective amount provides for analgesia or antihyperalgesia.
